(19) United States
(12) Patent Application Publication
(10) Pub. No.: US 2020/0227169 A1
(43) Pub. Date: Jul. 16, 2020

(54) Corrected BMI for Improved Assessment of Human Weight–Related Pathology

(71) Applicant: United States of America as represented by the Administrator of NASA, Washington, DC (US)

(72) Inventor: Steven A. Curtis, Dayton, MD (US)

(21) Appl. No.: 16/244,555

(22) Filed: Jan. 10, 2019

Related U.S. Application Data
(60) Provisional application No. 62/616,479, filed on Jan. 12, 2018.

(51) Int. Cl. G16H 50/30; G16H 20/60; A61B 5/00; A61B 5/107

(52) U.S. Cl. CPC G16H 50/30; G16H 20/60; G01G 19/4146; A61B 5/1072; A61B 5/4872

(57) ABSTRACT

A method and apparatus for correcting Body Mass Index (BMI) where a plurality of inputs, such as, subject's sex, mass, height, waist size, body temperature, average outside temperature, room temperature, and hours spent outside per day are stored to compute a corrected BMI (BMIC). The BMIC is in turn used to accurately minimize metabolic syndrome inflammation to extend life expectancy through a temporal psychologically stable human diet in a calorie rich environment.

### CROSS REFERENCE TO RELATED APPLICATIONS

Applicant claims priority to U.S. Provisional Patent Application No. 62/616,479, filed Jan. 12, 2018, entitled “A CORRECTED BMI FOR IMPROVED ASSESSMENT OF HUMAN WEIGHT-RELATED PATHOLOGY” and is hereby incorporated herein by reference in its entirety.

### ORIGIN OF THE INVENTION

The invention described herein was made by an employee of the United States Government and may be manufactured and used by or for the Government of the United States of America for governmental purposes without the payment of any royalties thereon or therefor.

### FIELD OF THE INVENTION

The present invention is related to a corrected Body Mass Index (BMI) and method of calculating same.

### BACKGROUND OF THE INVENTION

Long term secular increases in BMI in the U.S. and throughout the world pose a major threat to world health and affordable health care. High BMI and related metabolic syndrome drive many health related issues. Arguably, many seeming age related problems from diabetes to Alzheimer are in actuality driven by age related increases in BMI that peak in the 6th and 7th decades of life (afterwards there is a disease mitigated decline). NASA applications to extreme duration space missions and the need for healthful long term BMIs for human crew require a new stable approach to human diet in a calorie rich environment.

In best case scenarios, Diets have traditionally been associated with short term success followed with long term relapse and associated weight gain. More commonly, the diet fails even before weight targets are reached, often with a higher final BMI than at the diet start. Diet associated defects can be remedied through a proper examination of psychological instabilities, and how those instabilities can be mitigated by the Stability Algorithm for Neural Entities (SANE), which was the subject of a U.S. Pat. Nos. 8,041,661 and 8,095,485 each hereby entirely incorporated herein by reference. This psychologically and temporally stable diet solution, the Asymptotic Diet Algorithm with Psychological and Temporal Stability (ADAPTS), satisfies the SANE stability criteria in the specific case of total calorie consumption by several means: (1) the diet targets the Basal Metabolic Rate (BMR) of the desired BMI and by so doing both avoids a destabilizing starvation response that provides a large destabilization perturbation by the SANE stability criteria; (2) the BMR target provides a natural, low perturbation solution for the life-long eating levels at the end of the diet by providing an asymptotic approach to the final diet and BMI, and hence minimizing destabilizing perturbations; (3) the asymptotic approach continues the eating patterns under the BMR constraints over a long time and hence meets the criteria for adaptive perturbations under the SANE criteria, resulting in permanent behavioral modification.

On one test case, body mass was reduced from 230 lbs to 150 lbs over a 16 month period using an asymptotic BMR of about 1450 Kcal, which corresponds to a BMI drop from 33 to 22. In the same case, the caloric reduction originated from 2000 Kcal to 1450 Kcal. Note that the reduction is much smaller than typical diets, which are much more unstable. However, the long term solution is determined by the BMR calorie level consumed. Convergence criteria are BMI and waist size. Waist size when coupled to BMI gives a precise body composition indication (percent body fat) compensating for variations in muscle mass and frame size.

### SUMMARY OF THE INVENTION

The present invention is directed to a method and apparatus where a plurality of inputs, such as, subject's sex, mass, height, waist size, body temperature, average climate temperature, room temperature, and hours spent outside per day are stored to compute BMI_{c}. With the plurality of inputs, a subject's BMI is computed. The subject's BMI is used to determine the average population's waist size and percent body fat. The subject's waist size is used to compute a percent body fat. The subject's exposure to average outside temperature, room temperature, and hours spent outside per day are used to compute a correction factor to account for local seasonal climate temperatures. The subject's BMI, waist size, and percent body fat coupled with the average population's waist size and percent body fat, and the correction factor to account for local seasonal climate temperatures are all used to compute BMIc. Once BMI_{c} is determined, a stable psychological diet solution is achieved by reaching an optimal radiative efficiency level where chronological and physiological age are used to reduce deteriorating inflammations, metabolic syndrome and genetic predisposition to disease, in humans. The reduction of inflammation in a subject is achieved by properly managing the level of BMR in a subject. The stable psychological diet solution comprises of frequency initiated technique where a stable exercise program whose operating principles are directly derived from SANE. The stable exercise program starts and continues with the requirement that the program executed be performed daily. The stable exercise program has three stages: Frequency, Duration, and Intensity. These stages are listed not only in implementation order but also in priority order. The underlying rationale behind this staging is to minimize psychological instabilities which characterize most exercise and result in ultimate failure through pathways such as injuries and abandonment. The initial state is maintained until it is easily repeated on a daily basis, From this starting point, the duration of activities is gradually increased consistent with the daily repeat requirement until a target duration is obtained. In aerobic activity this would be a time interval. For resistance activity this would be a number of sets with a given number of repetitions per set. When the duration goal has been met and maintained long enough to be a stable daily pattern, then intensity can be slowly increased consistent with maintaining the frequency and duration requirements that have been set prior to the start of higher intensity training.

### BRIEF DESCRIPTION OF THE DRAWINGS

FIG. 1 is a chart depicting an empirical assessment of aging, comparing physiological age to chronological age.

FIG. 2 is a chart depicting the effect on aging due to BMI_{c} departures from optimal energy balance.

FIG. 3 is a chart depicting cross species comparison with tₚ normalized to human tₚ in mass (M), range, with human mass (MH).

FIG. 4 is a flow chart showing the general process by which to obtain the corrected BMI (BMI_{c})

FIG. 5 is the architecture of the computing system used to provide a user their BMI_{c}.

### DETAILED DESCRIPTION OF THE INVENTION

Asymptotic Diet Algorithm with Psychological and Temporal Stability (ADAPTS) approach is based on limiting the magnitude of any strong perturbations than can and have traditionally psychologically destabilized those wishing to permanently lose body mass. The phases are:

### Regulation

This step involves eating on a regular schedule and defining a meal structure that is followed every day. The structure can be typically 2-3 meals per day. The important part is to allow adaptation to a regular eating pattern that avoids any form of starvation response. No change in what or how much is eaten is made. Typical adaption times may be months.

### Substitution

With the regularized eating patterns adapted to under REGULARIZATION, the next step is to include low calorie, high volume foods as the first items eaten at each meal. The use of these foods should be staged in of a number of months to allow adaptation. Aside from the constraint that low calorie, high volume foods are eaten first at each meal, no requirements are made to remove any foods from the meals. The foods are chosen so that small low volume high calorie foods are not eaten when hunger can typically drive overeating before the hunger is attenuated. Also small variations in volume in low calorie, high volume foods have less of a weight gain impact. Typical time scale is months. The total calorie volume of the low calorie foods consumed is based on the BMR₍₁₎ as given in THE FIRST ASYMPTOTE IMPLEMENTATION as given below.

### Stabilization

The eating pattern established under REGULARIZATION and SUBSTITUTION is followed for several months until a stable weight is attained. During this period weight loss or gain attained is not as important as simply achieving a stable weight and a well-defined eating pattern which will form the basis for the actual caloric reduction(s) to be implemented in the next steps. This step is also of several months duration typically to allow full adaptation.

### First Asymptote Implementation

Since the subject is now eating a regular meal schedule and has a stable weight, the next step is to reduce the total calories eaten. The subject's Basal Metabolic Rate (BMR) at the subject's stable weight, BMR₍₀₎ and the BMR of the target weight of the first asymptote BMR₍₁₎ are computed:
For men: BMR=13.75 weight(kg)+5.003 height(cm)−6.775 age+66.5.
For women: BMR=9.563 weight(kg)+1.850 height(cm)−4.676 age+655.1.

The difference, dBMR=BMR₍₀₎−BMR₍₁₎, gives the number of calories to be removed to attain the desired weight. It is noted that the subject never eats less than will be consumed at the target weight, which as long as such target yields a BMI_{c}>18.5 should result in no starvation response and hence no psychological instability. A recommended target is a BMI_{c} in the 24-25 range. As an example a male subject at age 60, height 69.5 inches, and a body weight of 235 lbs would require a dBMR of 400 calories/day. This stage, depending on the magnitude of the weight loss required, can take from months to a year plus.

### Second Asymptote and Subsequent Implementation

If additional weight loss is needed, after the first asymptote is attained, a second asymptote can be entered on by a caloric reduction given by:

BMR_{(first asymptote)}−BMR_{(second asymptote)}

This could for example target a lower BMI_{c} range (e.g. 20-22) or simply be a needed refinement to the first asymptote to achieve the initial first asymptote target BMI_{c}. As an example, a male subject with a height of 96.5 inches and a weight of 170 would require a second dBMR of 160 calories/day. Third and subsequent asymptotes can be implemented as needed. The final asymptote will be the final diet for the subject (subject to aging).

After the final asymptote weight is achieved, the subject must remember that the BMR decreases with increasing age. For example a 60 yr old male with food consumption equal to that needed to maintain a weight of 145 lbs at 69.5 inches height would gain 35 pounds if he continued to eat at the same level until age 90. So as the subject ages additional asymptotes will be required to maintain the target BMI_{c}. To avoid this consequence the subject will need to reduce caloric consumption by about 200 calories/day by age 90. Thus is a slow process that can easily be accommodated within the ADAPTS framework.

In the formulation presented here, the assumption is that the net physical activity does not vary. Exercise is an excellent process for health, but not a psychologically stable one for weight loss since variations in exercise owing to schedule, injury, etc., can produce a continually varying caloric target and hence instability. The stable approach to the inclusion of physical activity, either high or low, is to adjust the diet whenever there is a large spike in exercise for that occurrence. An excellent rule of thumb, from a colleague's grandmother who lived in India and lived to 104, is to only eat above your established asymptote when sustained physical activity requires it.

### Impact of Adapts on Aging Through BMI Reduction

A psychologically stable approach to BMI management that is scalable to entire populations with minimal capital investment can achieve a dramatic extension of life expectation past the age of 70. The specific goal is to lower mean population BMI to the lower 20's. This would represent a new direction for worldwide populations that are now trending toward the low 30's and beyond. Major consequences of this reduction in population BMI would be an increase in productive years of employment, increased lifetime savings and a reduction in probable lifetime health care costs. As a result, the threat of rising health care costs bankrupting the future and precluding needed levels of technology investments may be averted.

Since 1850 in the US, life expectation from age 70 has increased only 4 years for males and 5 years for females. Similarly, life expectation from age 80 has only increased 2 years and 3 years respectively for males and females. This is in stark contrast to life expectation from birth, which has increased 37 years for males and 40 years for females. Much of these differences between life expectancy from birth and that from older age stems from the success of disease mitigation in the young as opposed to the more difficult processes associated with the aging process. Aging may be viewed as a cumulative effect of inflammation integrated over a lifetime. A hallmark of inflammation is metabolic syndrome which is directly related to BMI. In addition to BMI related inflammation, there is also inflammation owing to non-BMI specific causes such as genetic predisposition to disease. BMI characteristically increases sharply with age. For example, a 20 year old male with a BMI of 21. is at the 28th percentile, whereas a 60 year old male is at the 5^{th} percentile. In addition, a 20 year old male with a BMI of 31 is at the 91^{st} percentile, whereas a 60 year old is at the 75^{th} percentile. At the threshold for metabolic syndrome (BMI=25), a 20 year old male is at the 60^{th} percentile, whereas at age 60 is only at the 30th percentile. Similar results are obtained for females. The pronounced upward drift in BMI as a function of age has major implications for future health care costs. This a global problem with many nations sharply trending toward average BMI's of 30. If anything, the problem is probably understated based on a BMI_{c} which accounts for relative radiative efficiency based on body geometry and composition.

Two commonly accepted parameters in the aging process are: (1) chronological age or actual elapsed time age, and (2) a measure of physiological decline with age or physiological age. These two parameters can be plotted orthogonally to form a 2 dimensional space with axes: physiological age, tₚ, and chronological age, t_{c}. For physiological age, there is a commonly accepted maximum for humans of about tₚ≈85. We adopt t_{p max}=85 for the purposes of this discussion. As a basis, we note that if all cardiovascular disease, the largest avoidable form of mortality, were eliminated, the average life expectation would increase by 7 years, or a value of about 85 years. It is noted that t_{p max} can be either greater or less than t_{c max,} the maximum chronological age attained, depending on a given human's aging rate(s). Notionally, for an average person, defined as BMI≤25, the aging profiles follows t_{c}=tₚ, a line starting at the origin of the t_{c}⊗tₚ space with a slope of unity. The aging implicit in tₚ has two components: a chronological and achronological aging process. A chronological aging process selectively ages humans to maximize longevity where t_{c}≤75. On the other hand, an achronological aging process is solely a function of inflammatory levels and their effects on human deterioration rates where t_{c}≥75. Inflammation is key to the chronological aging process and results in more rapid aging than that driven by t_{c} alone.

In general, we can relate tₚ to t_{c}:

$$\begin{matrix} \text{?} = \text{?} ( {C}^{*} + {I}^{*} ) dt & ( 1 ) \\ \text{?} \text{indicates text missing or illegible when filed} & \end{matrix}$$

where I* is the normalized inflammation rate impact and C* is the normalized chronological aging rate, which vanishes for tᵣ>75.

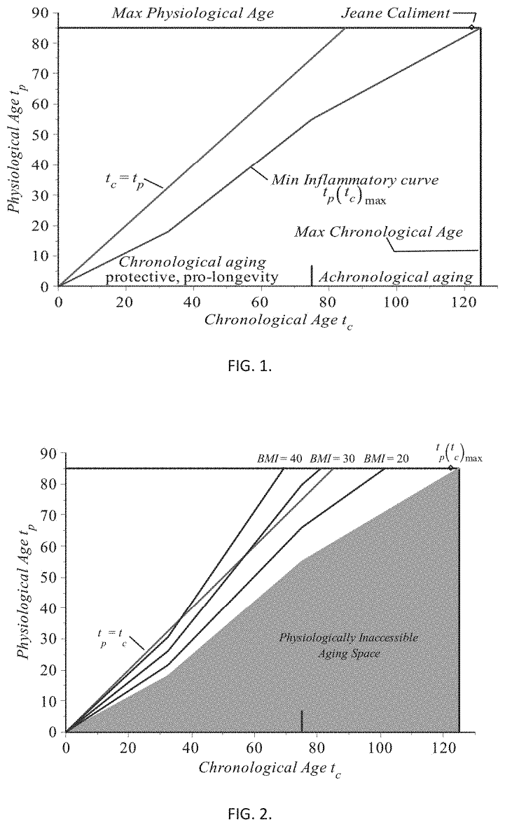

To evaluate this integral for tₚ, we introduce the concept of a minimum inflammatory curve, which maximizes t_{c} for a given tₚ. We call this tₚ(t_{c})ₘₐₓ. At the low end of this curve, observational data is used. For example, the oldest a person is when still asked for identification for alcohol is 32.5 where the legal age is 18. A second point can be obtained by noting that the youngest a 75 year-old may appear is about 55. Physical appearance is taken here as an important indication of overall physiological aging since skin is an external organ that often gives indications of the aging rate of internal organs. The intercept of the tₚ(t_{c})ₘₐₓ curve with the maximum physiological age t_{p max} can be obtained from the extensive records of super centenarians. The super centenarians, t_{c}>110 are extremely rare with less than 100 out of a documented population of more than 10⁹. The t_{c max} observed is at present 122.5, achieved by Jeanne Marie Calment of France. No other documented age has exceeded 120. Very few super centenarians live to t_{c}=115. Given these data, it appears safe to assume t_{c max}=125. Per FIG. 1, the three points discussed here determine the tₚ(t_{c})ₘₐₓ curve. To evaluate the integral for tₚ as a function of BMI, we note first that we are using BMI_{c} as derived in A CORRECTED BMI FOR IMPROVED ASSESSMENT OF HUMAN WEIGHT-RELATED PATHOLOGY as given below, BMI_{c} derives corrections to BMI based on the relationship between BMI and radiative efficiency in humans. There are two parts to the departure from the tₚ(t_{c})ₘₐₓ curve: BMI_{c related} and BMI_{c independent}. The BMI_{c related} component is taken as linear in BMI_{c}. Hence we can write equation (1) as:

$${t}_{p} = {{t}_{p} ( {t}_{c} )}_{max} ( 1 + ( ( \frac{B M {I}_{C} - 20}{40 - 20} ) θ ( B M {I}_{C} - 20 ) + ( \frac{20 - B M {I}_{C}}{20 - 12} ) θ ( 20 - B M {I}_{C} ) ) {g}^{*} + {g}^{* ′} ) \qquad ( 2 )$$

where θ is the unit step function

$$θ ( α ) = \{ \begin{matrix}0 & α < 0 \\ 1 & α \geq 0\end{matrix}$$

and g* and g*′ are respectively the BMI related and the non-BMI related aging rates.

From the BMI linkage to radiative efficiency, stress increases for departures above (inefficient) or below (overefficient) an ideal BMI, which we take to be BMI_{c}=20. For the BMI_{c} in the BMI dependent terms, we have taken a range of 40 (morbid obesity) for the under efficient radiation case and 12 (near starvation) for the overefficient case. With these values, values for g* and g*′ were derived by fixing the BMI_{c}=25 line to correspond to tₚ=t_{c} line. This yields

g*=0.5 g*′=0.2 (3)

We note that for g in the case of extreme inflammation owing to childhood cancers g*′→85 and have for some parts of the population much higher g*′ are possible owing to severe disease onset. Hence, in general g* is equal to g*′(t_{c}). Using equation (2) we can calculate the aging curves for BMI_{c}=40 (obese), BMI_{c}=30 (overweight) and BMI_{c}=20 (ideal). These are shown in FIG. 2. In the case of super centenarians, g*′=0 and for BMI_{c}=20 although g* may also be small for them and hence reduce BMI related aging effects.

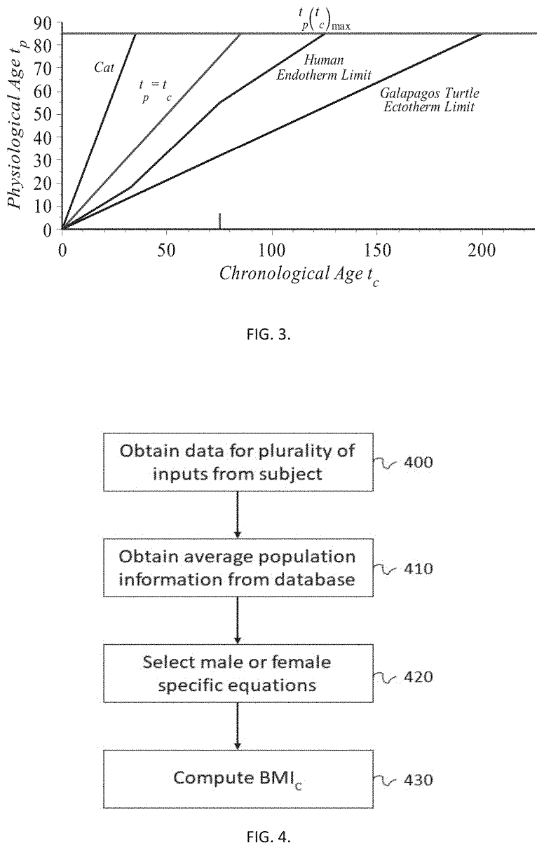

Finally per FIG. 3, we examine minimum aging curves, tₚ(t_{c})ₘₐₓ for other species to put the human result in context. We show results for domestic cats (maximum documented age t_{c}=35) for which we take t_{c max}=40 and for Galapagos tortoises (maximum documented age t_{c}=180) for which we take t_{c max}=200. We have restricted the mass range for comparison to within a factor of 10 of human mass. This keeps the comparison within a similar mass scaling regime and avoids scale related anomalies such as mice or whales. Most importantly the Galapagos tortoise represents a limiting ectothermic case. Since ectotherms have much lower metabolic rates and hence much lower inflammation rates, they give an indication of the maximum life extension possible if the inflammation levels were reduced to that of an optimally aging ectotherm. This underlines the difficulty of any extension of the maximum human lifespan beyond 125 assumed here High human endothermic metabolic rates and the related high energy requirements for the human brain, effectively preclude adding any longevity enhancement anywhere near that of tₚ(t_{c})ₘₐₓ for a comparable ectotherm. Given the comparable maximum cell divides of both ectotherms and endotherms.

We now turn to the economic implications of the BMI_{c} curves and the related economic impacts of ADAPTS. From FIG. 2, the t_{c} at which tₚ=70 is reached in the t_{c}=80 for BMI_{c}=20, t_{c}==66 for BMI_{c}=30, and t_{c}=58 for BMI_{c}=40. Since a physiological age of 70 is the present projected social security retirement age (reflecting the population on the tᵣ=tₚ curve), a person with a BMI_{c}=40 would lose 12 years of productive work, with BMI_{c}=30, 4 years, and with BMI_{c}=20, would gain 10. Since saving accruals are exponential owing to interest compounding, the additional years of productivity help to offset the cost of typical medical expense surge as life expectance is approached, as well as overall retirement casts. ADAPTS, with a convergence criterion based on BMI_{c}, which adaptively changes eating patterns to achieve an optimal BMI_{c} in a psychologically stable manner is ideally suited to provide financial stability given the worldwide aging demographics of the next several decades. The critical need to a near term start toward a solution is outlined in a related white paper.

### Frequency Initiated Technique for a Novel Exercise Stability System (Fitness)

FITNESS is not intended as a methodology to attain peak physical performance, since that is by a definition and construction an unstable condition from which there is eventual collapse—the off seasons of all competitive high performance athletics. Rather FITNESS is designed to provide a stable and sustainable level of physical performance that would greatly enhance the long term health of the bulk of the population while at the same time provide a methodology for off season conditioning for high performance competitive athletes. High performance athletics is characterized by the use of high inflammation training methodology focusing on intensity that produces the most rapid although unsustainable gains. Indeed studies in which subjects consume classic antioxidants which reduce inflammation levels show characteristically significantly lower short term gains than subjects not taking antioxidants. This has been shown to be true both for endurance and for strength. Despite the attraction of more rapid short term gains, there are definite consequences of enhanced inflammation both short term and long term. The short term consequence is that the sustained higher inflammation rates lead to a near term collapse from the high conditioned state and, hence, lacks longer term stability. The long term implications are more disturbing in that time integrated inflammation drives the physiological aging process beyond the normal programmed aging sequence. It can be truly said that high performance athletes are trading their future health for their present performance. This trade can be minimized with FITNESS in that it is a minimally inflammatory approach to physical performance where the net inflammation from FITNESS is negative as opposed to positive. This is achieved by strongly controlling the intensity levels of exercise in contrast to other methodologies which put an emphasis on intensity and, hence, assure a large net positive inflammatory response which undermines the exercise programs long term stability. FITNESS minimizes the High Inflammatory Response (HIR) of intensity focused exercise assuring long term exercise program stability.

Another more insidious result of intensity focused exercise is the Exercise Intensity Response (EIR) which is endorphin mediated and, hence, addictive. With high intensity exercise, EIR will result in an attempt to increase endorphin levels as receptor initial response declines as in all additive processes. As a consequence, HIR will rise to level of high chronic inflammation with associated high incidence of chronic injuries and eventual exercise program collapse.

FITNESS stabilizes against EIR and HIR by reversing the order of exercise priorities compared to most programs. Instead of placing intensity first, it is last. The highest priority in FITNESS is to maintain frequency of exercise. This is followed by duration of exercise. Last is intensity. In FITNESS, frequency is based on a 24 hour recovery cycle that is implicit in all human activities. Any effort requiring longer, multiple day recovery is long term unsustainable and unstable. Hence, the overarching constraint on FITNESS activities is that they must be reproducible on a 24 hour cycle. This also minimizes any deconditioning that may result from longer recovery periods, particularly in older populations which rapidly decondition. In practice this means FITNESS is implemented as a 7 day a week program designed to become part of daily life much as eating and sleeping,

When initiating FITNESS with endurance and strength training, the initial durations and intensities are started well below capabilities. Literally at start, the FITNESS program is just going through the motions to condition the subject to daily frequency alone. The FITNESS program is optimally fully implemented indoor, reducing the effects of weather on training. However, outdoor components are acceptable so long as there exist established indoor options in inclement weather. FITNESS is designed to have both an endurance and a strength component.

In the case of weight or resistance training, starting levels should be one half of levels that require concentrated effort to compete and are performed for only 1 set of 6-8 repetitions. Similarly, for endurance exercise, the intensity level should be where a normal conversation can be conducted while exercising with a duration of less than one half an hour. As a specific example for a spinning bike, resistance levels are set for minimum effort for a duration that is comfortable to the subject of less than 30 minutes. The frequency focused initialization of FITNESS continues at low duration and intensity until the subject has sustained a stable daily program. Typically this period will last for 1-2 months.

After the daily frequency of exercise is stabilized at low duration and intensity levels, the duration implementation phase of FITNESS is entered. In this phase, the duration of both the endurance and strength components are increased to final target levels with no increase in intensity and while maintaining daily frequency. For endurance, this means gradually increasing the duration time of the exercise up to one half to one hour. For strength, this means increasing the number of sets from 1 to 3 or 4. This phase is usually completed in a month.

After the completion of the duration phase, the subject is now exercising daily with the target duration for endurance and strength training. At this point the subject is stable enough to begin increasing intensity while maintaining established daily frequency and achieved levels of duration. Endurance intensity is generally increased by speed and resistance. For example, on a spin bike, the spin rate and the resistance setting. Strength intensity is achieved by increasing resistance, most often the poundage of weights used. The intensity increase phase generally will last from 6 months to one year. During this time the subject will saturate at their capacity levels for both strength and endurance. Although this may mark the quantitative end of gains in the program, qualitative gains in body composition and health will continue as the typical full adaptation time is 2 to 3 years. Peak sustainable physical performance can then be maintained for a lifetime.

We note that for all three phases of FITNESS, the implementation time scales can be much shorter for conditioned athletes who have well established workout routines which can be modified directly into the FITNESS framework. Also reentry into the program after an absence will also be faster owing in experience. Removal of senescent cells and reduced inflammation with negative disease resistance consequences verifies ADAPTS/FITNESS models of aging as time integrated inflammation with greater longevity with reduced native inflammation rate but with the condition that earlier life has reduced exposure to disease/injury for which reduced inflammation is a liability.

In the specific cases of ADAPTS and FITNESS, the goal is to implement rule sets over adaptive time scales which will provide for stable behavior in the areas of diet and exercise, respectively. For both ADAPTS and FITNESS, this requires a detailed examination Of stability conditions guided by SANE. This examination resulted in a set of rules for ADAPTS and FITNESS which provide for a psychologically stable implementation of diet and exercise programs,

In the case of ADAPTS, the key is the recognition that the main destabilizing influence is triggering the Human Starvation Response (HSR). In the case of FITNESS, the key is the recognition of the destabilizing role of Exercise Intensity Response (FIR) and High Inflammatory Response (HIR). In both cases, the SANE result would be for psychological instability based on the large changes in psychological element interplay due to the large perturbations imposed. Hence, for ADAPTS, a set of rules are developed consistent with HSR suppression. Similarly, for FITNESS, a rule set are developed consistent with EIR suppression.

HSR may be characterized as an endorphin mediated behavior which elicits pleasure when eating while hungry. There is an obvious evolutionary advantage to this behavior in that it provides incentivized motivation to find and eat food and, hence, lowers the possibility of starvation. However, HSR may be subverted by manipulating the response to hunger by (1) serially starving and then overeating resulting in a secular gain in body mass, or (2) continuouly starving to ever greater degrees wherein a decreasing amount of food produces an ever more acute endorphin response. In the case of (1), therein lies the basis of the continuous surge in body mass and related metabolic syndrome disease that constitute a global pandemic. In the case of (2), the driver of anorexia nervosa revealed, a potentially rapidly fatal condition. Both (1) and (2) constitute strongly addictive behaviors which require a SANE mediated rule set applied over adaptive time scales to correct.

EIR may be characterized as an endorphin mediated behavior which elicits pleasure due to increasing intensity of exercise. Unfortunately if not checked, EIR can result in serious chronic injuries as well as in an aversion response to exercise as a result of later pain and injury after exercise and the endorphin levels subside. Before collapse, however, a prolonged spiral of increasing exercise intensity followed by pain and injury responses can ensue. This is a highly inflammatory process that obviates the health gains to be obtained by exercise.

The structure of ADAPTS is hence derived from HSR minimizing sequence of rule applications, which are slowly implemented using SANE stability criteria of using of changes which produce a small change in the psychological state vector. This allows a departure from one stable psychological state vector value to another, as the diet is implemented. The time sequence of applied rules is as follows:
(1) Regularization of eating times with eating to satiation at each meal.
(2) Substitution of higher protein sources as well as lower calorie density foods.
(3) Restriction of calories to level of target weight BMR (Basal Metabolic Rate)

The structure of FITNESS is correspondingly derived from an HR minimizing sequence of rule applications, which are again slowly implemented using the same SANE considerations as for ADAPTS. This will again allow the departure from one stable psychological state vector to another, as the exercise program is implemented. The time sequence of applied rules is as follows:
(1) Frequency of exercise
(2) Duration of exercise
(3) Intensity of exercise.

The metric for measuring progress and eventual success in both ADAPTS and FITNESS is the BMI_{c}.

### BMI_{c} for Improved Assessment of Human Weight-Related Pathology

BMI can be directly related to human radiation inefficiency as measured by volume to surface ratio (VSR) by assuming that a normal population's waist size scales with height. Identifying the VSR as the underlying link to the overall environmental stress on a human, we derive correction factors to the BMI based on relative waist size, relative percent body fat, and average external environmental temperature. The first two of these corrections are gender specific. The last of these would appear to apply most to those in pre-industrial societies with significant exposure to average outside climate temperatures. A simple formula resulting from these relationships is given for computing a corrected WI, acting as a more accurate measure of obesity closely tied to waist size. Illustrative examples are given.

Although many in the literature have assumed that WI is something of a fluke data ordering parameter for health standards, others have thought that it was fundamentally a scaling parameter for overall metabolic rate excess (how fast an endothermic animal is slow cooking itself due to internal heat generation).

We can show that BMI scales with VSR. The bigger the VSR, the greater the cooking rate thermal stress. There is a lower limit to VSR determined by over radiation and, hence, stress from over cooling as compared to under radiation and, hence, stress from overheating. Thus,

|VSR|ₘᵢₙ<VSR<|VSR|ₘₐₓ (4)

far healthy VSR. Based on present literature the corresponding BMI range is 18.5 to 24.9. The threshold for anorexic underweight is 17.5, for overweight 24.9, obesity 30, and morbid obesity 40. Now

$$V S R = \frac{V}{A} \qquad ( 5 )$$

for a given V and surface area A, and

$$B M I = 703 \frac{m}{{h}^{2}} \qquad ( 6 )$$

where m is the body mass (lbs) and h is the height (in), and 703 is the conversion factor used to scale from imperial units to metric units.

### BMI Varition and Correction for Waist and Density Within Populations

If we approximate a human as a cylinder and assume that the waist, W (the circumference of the cylinder) scales as αh

W=αh (7)

where α is waist to height ratio, and h is the height, then A scales as αh² and

$${h}^{2} = \frac{A}{α} \qquad ( 8 )$$

since

m=Vρ (9)

where ρ is the average density in imperial units, then

$$B M I = \frac{m}{{h}^{2}} = α ρ \frac{V}{A} = α ρ VSR \qquad ( 10 )$$

Now ρ scales as the volumetric change for fixed h, since

V=α²h³ (11)

### Where

$$α = \frac{A}{{h}^{2}} \qquad ( 12 )$$

Thus for fixed h and m (fixed BMI), ρ scales as

$$( \frac{1}{{α}^{2}} ) ( \frac{{ρ}_{1}}{{ρ}_{0}} )$$

where ρ₁ is the absolute density increase relative to the average density, p₀, (e.g. more muscular versus less means a higher density). Thus

$$α ( \frac{{ρ}_{0}}{{ρ}_{1}} ) B M I = VSR \qquad ( 13 )$$

In the case of an individual with body density, ρ₁, and waist to height ratio, α₁, matching BMI population values for those parameters ρ₀ and α₀, respectively,

$$\frac{{ρ}_{0}}{{ρ}_{1}}$$

is one and we have

VSR=α₀BMI (14)

or in general, we can write for any individual with α₁ and ρ₁:

$$VSR = {α}_{0} ( \frac{{α}_{1}}{{α}_{0}} ) ( \frac{{ρ}_{0}}{{ρ}_{1}} ) B M I = {α}_{0} B M {I}_{c} \qquad ( 15 )$$

### Therefore

$$B M {I}_{c} = ( \frac{{α}_{1}}{{α}_{0}} ) ( \frac{{ρ}_{0}}{{ρ}_{1}} ) B M I \qquad ( 16 )$$

Hence, BMI is directly related to volume to surface ratio VSR. This physically explains the significance of BMI as a predictor of overall metabolic stability and, hence, health. Since the VSR is the real physical parameter, BMI can give a wrong answer for very muscular humans (lower than average a or relative waist size and/or higher absolute density ρ₁ resulting from more muscle). It can also give a wrong answer for very unfit humans, since then a given BMI will give a higher VSR. Since fat is 0.9 g/cc, and muscle is 1.1 g/cc, there is an effect as one goes from high to low percent body fat and, hence, low to high ρ₁, but it is limited by the overall departures from the percent body fat that is the norm (usually less than 30%). The waist parameter α is a stronger player here since more than 10% variations are possible.

For illustration, an individual with a waist size of 32 in rather than 38 in for a BMI of 30 would have an effective BMI of (32/38)×30 or about 25, at the high end of normal as opposed to borderline obese. If one further corrects for absolute density changes for such a relatively muscular individual (the body fat decreasing from 30% to 10%, the equivalent of 20% of the body mass), and that body mass increased by 22% (density increase from fat to muscle), the overall absolute density increase is 0.2×0.22 or about 0.04. This would further reduce the effective BMI to 25/1.04=24, which is totally in a normal range. It should be noted that very few individuals are this athletic. At the other extreme consider a very unfit individual with a BMI of 24, but a waist of 42 in instead of 34 in, and a 40% body fat instead of 20%. Then as before the effective BMI is 24×(42134)×1.04=30, or the individual is actually clinically obese despite having a BMI in the normal range.

Both case here are extreme, but serve to show the range of variations. For more modest departures, there can be significant health implications for those with BMI values plotting near under-weight and over-weight boundaries. In summary, high BMI produces a less favorable VSR for heat rejection and, hence, results in more internal heat and related thermal induced breakdowns. For sufficiently low BMI, the human fails to maintain a sufficiently high internal temperature which also results in stress and breakdown. The classical BMI can be corrected from its departures from correctly tracing the physical implications of the VSR by noting either anomalous waist size or percent body fat. We note that for humans the lower limit to VSR is driven in part by brain energy needs (20% total for humans). The implication is that the attempts to extend life by the more extreme calorie restriction that has been successful for some animals will not work for humans given the literal brain drain and resulting physical and psychological instabilities from going below |VSR| or the corrected |BMI_{c}|ₘᵢₙ. For humans, the best strategy is to remain in the optimal VSR or BMI_{c} that have empirically population derived upper and lower bounds.

### Temperature Effects

There still remains the need to account for local seasonal climate temperature effects on BMI. The present results reflect the general radiative efficiency for a given body geometry. In this discussion, SVR, the surface to volume ratio, is the reciprocal of VSR. When external temperature effects are included, total radiation from a body will scale as SVR (T_{B}⁴−T_{E}⁴). For |T_{B}−T_{E}|<<T_{B} or T_{E}, we have approximately

VSR=4SVR|T_{B}−T_{E}|=4SVRΔT (17)

where T_{B} is the body temperature and T_{E} is the environment temperature. We note that radiative heating and cooling dominates for humans. A given human will spend time fraction, β, outside and (1−β) inside, where T_{ES} is the average room temperature, T_{EH} the hot climate average outside temperature and T_{EC} the cold climate average outside temperature. We have the hot climates' correction factor T′_{EH} for BMI:

$${T}_{EH}^{′} = \frac{1}{( 1 + β ( ( \frac{Δ {T}_{EH}}{Δ {T}_{ES}} ) - 1 ) )} \qquad ( 18 )$$

where

$$\begin{matrix}β = \frac{time ( hrs )}{24 hrs} & ( 19 ) \\ Δ {T}_{EH} = {T}_{B} - {T}_{EH} & ( 20 ) \\ Δ {T}_{ES} = {T}_{B} - {T}_{ES} & ( 21 )\end{matrix}$$

and for cold climates' correction factor T′_{EC}:

$${T}_{EC}^{′} = \frac{1}{( 1 + β ( ( \frac{Δ {T}_{EC}}{Δ {T}_{ES}} ) - 1 ) )} \qquad ( 22 )$$

where

ΔT_{EC}=T_{B}−T_{EC} (23)

Note for most individuals, the external temperature is well approximated by room temperature so the correction factor is unity. However, for those living in non-industrial societies, β is non zero. For example, temperature values could be 3121K for T_{B}, 295K for T_{ES}, 305K for T_{EH}, and 273K for T_{EC}. We also assume that β is 0.333 (8 hours outside per day) for cold climates 0.6667 (16 hours outside per day) for hot climates. The variation of β between hot and cold climates corresponds to observed human activity in those regions.

Then for hot climates, would be equal to T′_{EH} would be equal to 1/0.83 or 1.2, and a BMI of 24 would yield a BMI_{c} of 29. Thus an acceptable BMI individual with substantial heat exposure would have a climate BMI_{c} that is borderline obese. For cold climates: T′_{EC} would be equal to 1/1.4 or 0.7 and a BMI of 35, which would be significantly obese, would have a cold climate BMI_{c} of 24.5, which is in the healthy normal range. Obviously, the calculation here has simplifications; however, it serves to show that there exist large BMI_{c} climate corrections to BMI that could substantially effect the health assessment of overweight or underweight status based on BMI. However, the calculations correctly yield the observed BMI distributions of native populations in non-industrial societies as a function of climate.

### Derivation and Illustrations

We have shown that BMI scales directly with volume to surface ratio and is a measure of radiative inefficiency for humans. The physics is completed by also incorporating the environmental temperature effects of the radiation process to this inefficiency. The resulting BMIc:

$$B M {I}_{C} = B M I \frac{{T}^{′} ( \frac{{W}_{1}}{{W}_{0}} )}{( \frac{1}{{ρ}_{0}} ) ( 1 \frac{lbs}{{in}^{3}} - \frac{( {P}_{1} - {P}_{0} )}{100} ( {ρ}_{M} - {ρ}_{F} ) )} \qquad ( 24 )$$

For the accuracies needed here, we can allow p₁ to be equivalent to 1.01 for the average human. Waist, W₁, is substituted for αh. P₁ is the individual's proportion of body fat and P₀ is the population proportion of body fat for specified BMI. W is the corresponding population average waist. T is the climate correction factor that can either equal T′_{EH} for hot climate, or T′_{EC} for cold climate. We note that gender specific BMI corrections enter P₁ and P₀. Since P₀ is substantially larger for females (F) than males (M) (ρ_{M}−ρᵣ has a value of 0.22 lbs/in³ (see equation 24)), the gender correction can be large. The W₀ is also gender specific; so we have, as shown for initial BMIs of 20, 25, and 30:

$$B M {I}_{C} ( M , F ) = B M I \frac{{T}^{′} ( \frac{{W}_{1}}{{W}_{0} ( M , F )} )}{( \frac{1}{{ρ}_{0}} ) ( 1 \frac{lbs}{{in}^{3}} - \frac{( {P}_{1} ( M , F ) - {P}_{0} ( M , F ) )}{100} ( 0.22 \frac{lbs}{{in}^{3}} ) )} \qquad ( 25 )$$

where BMI_{c}(M,F) is the gender specific corrected BMI. For most of our extended examples below we will use case 1; a male with BMI of 21.3, P₁ of 0.08, W₁ of 29 inches, h of 69.5 inches, and α (or waist to height ratio, WHR) of 0.42. Additionally, note that waist and height measurements are given in inches and weight in pounds in this discussion, unless otherwise specified. Typical a values are shown in Table 1. A healthy cutoff, for a BMI of 25, would be 0.50. Note that although waist is gender dependent, waist normalized by height is gender independent or equivalently waist ratios are gender independent.

TABLE 1
Typical α values

| Population | α |
|---|---|
| Barbie Doll | 0.25 |
| Ken Doll | 0.36 |
| College Swimmers | 0.43 |
| BMI of 20 | 0.43 |
| BMI of 25 | 0.50 |
| BMI of 30 | 0.57 |

P₀(M), the minimum proportion of body fat essential for health is 0.04 in males, and P₀(F) is 0.10 in females. A healthy proportion of body fat range is 0.15 to 0.18 in males and 0.20 to 0.25 in females. Athletes generally range in proportion of body fat from 0.05 to 0.12 and 0.10 to 0.20 for males and females, respectively, and the physically fit proportion of body fat range from 0.06 to 0.25 and 0.14 to 0.31, respectively. Consider the BMI for the case of a standard healthy range for the male (case 1) above where weight was 145 lbs, α is 0.46, P₀ is 0.15, and T′ is unity. Hence, BMI_{c} is (21.3×(0.42/.46)/((1/1.01)(1-(0.08−0.15)×0.22))=19.1.

The gender specific percent fat calculators, P % (M/F), used in the following equations are classic ones which make assumptions about minimal body composition, for males and females, respectively, where the first term in parentheses refers to average height (in inches), the second refers to waist size (W) in inches, and the third term to weight (m) in lbs:

$$\begin{matrix}{P}_{\%} ( M ) = 100 \frac{( - 98.42 + 4.15 W - 0.082 m )}{m} & ( 26 ) \\ {P}_{\%} ( F ) = 100 \frac{( - 7 \; 6.76 + 4.15 W - 0.082 m )}{m} & ( 27 )\end{matrix}$$

where P % (M) is the percent proportion body fat in a male subject, and P % (F) is the percent proportion body fat in a female subject. Another fat calculator used by the U.S. Navy considers neck and hip circumferences as well as waist and height.

The relationship between a and BMI is linear. Below, we derive the intercept and slope from plotting a typical range of subject's waist (αh) and weight data from NHANE WHR data above, in order to calculate P₁ and P₀ for BMI_{C}. For the relationship between a and BMI, we have

$$α = BMI ( \frac{d α}{dBMI} ) + {BMI}_{0} \qquad ( 28 )$$

with slope

$$( \frac{d α}{dBMI} )$$

of 0.014 and intercept (BMI₀) of 0.15. We now have a way of calculating both the standard waist, the standard percent body fat and the actual percent body fat.

The following example shows how a plurality of inputs, such as, subject's sex, mass, height, waist size, body temperature, average outside temperature, room temperature, and hours spent outside per day are stored to compute BMI_{c}. Using case 1, the first step is to calculate the subject's BMI from h (height in inches) and m (weight in lbs) with equation 6.

$$BMI = \frac{145 lbs}{69.5 in} = 21.1$$

Next, determine the average population waist to height ratio, α₀, with equation 28,

α₀=(21.1)(0.014)+0.15=0.445

Next, the male's average population waist size, W₀(M), is determined with the subject's height of 69.5 inches, the average population waist size to height ratio, α₀, and equation 7,

W₀(M)=(0.445)(69.5 in)=30.96 in

Next, the male's average percent body fat, P₀(M), is determined with the subject mass and the male's average population waist size, W₀(M), and equation 26,

$${P}_{0} ( M ) = 100 \frac{( - 98.42 + 4.15 ( 30.96 in ) - 0.082 ( 145 lbs ) )}{145 lbs} = 12.53 \%$$

Next, the subject's percent body fat, P₁, is determined with the subject mass and waist size, W₁, and equation 26,

$${P}_{1} ( M ) = 100 \frac{( - 98.42 + 4.15 ( 29 in ) - 0.082 ( 145 lbs ) )}{145 lbs} = 6.92 \%$$

Finally local seasonal climate temperature effects is evaluated where during the summer for example the hot climate average outside temperature, T_{EH}, is 305K, the average room temperature, T_{ES}, is 295K, the subject's body temperature, T_{B}, is 312K, and the time spent outside by the subject during the summer is 16 hours. Thus, the hot climates' correction factor T′_{EH} for BMI can be calculated using equation 18-21:

$${T}_{EH}^{′} = \frac{1}{( 1 + 0.66667 ( \frac{( 312 K - 305 K )}{( 312 K - 295 K )} - 1 ) )} = 1.645$$

$$where$$

$$β = \frac{16 hrs}{24 hrs} = 0.66667 ( time fraction )$$

$$Δ {T}_{EH} = 312 K - 305 K$$

$$Δ {T}_{ES} = 312 K - 295 K$$

The corrected BMI, BMI_{c}, is then calculated with equation 25 for a male (M):

$${BMI}_{C} ( M ) = BMI \frac{{T}_{EH}^{′} ( \frac{{W}_{1}}{{W}_{0} ( M )} )}{( \frac{1}{{ρ}_{0}} ) ( 1 - \frac{( {P}_{1} ( M ) - {P}_{0} ( M ) )}{100} ( 0.22 \frac{lbs}{{in}^{3}} ) )}$$

$${BMI}_{C} ( M ) = ( 21.1 ) \frac{( 1.645 ) ( \frac{29 in}{30.96 in} )}{( \frac{1}{1.01 \frac{lbs}{{in}^{3}}} ) ( 1 \frac{lbs}{{in}^{3}} - \frac{( 6.92 \% - 12.53 \% )}{100} ( 0.22 \frac{lbs}{{in}^{3}} ) )} = 32.4$$

The subject's BMIc accounting for the plurality of inputs corrects a BMI of 21.1 to 32.4.

The method of the present invention may be illustrated with the steps shown by the flow chart in FIG. 4. In step 400, data are obtained for a plurality of inputs, such as, subject's sex, mass, height, waist size, body temperature, average outside temperature, room temperature, and hours spent outside per day. In step 410, a database containing average population information such as male & female average densities, average waist to height ratio, and average human density is readily available for further computations of Milk. In step 420, based on the inputs provided by step 400, male or female specific equations, and hot or cold climate equations are selected for further computations of BMI_{c}. In step 430, the BMI_{c} is processed based on the inputs obtained from step 400, database information from 410, and equations from 420.

The flow charts in the figures provided herein are not meant to imply an order to the various steps, since the invention may be practiced in any order that is practical. For example, one may first obtain average, population information first before proceeding with the rest of the step provided in FIG. 4.

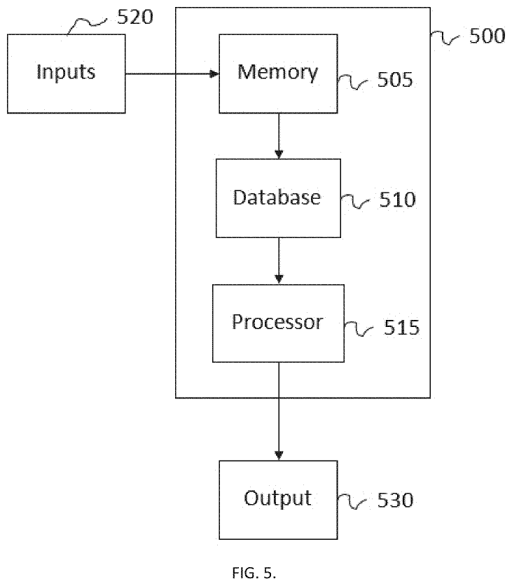

An apparatus for assessing BMI_{c} of the present invention is illustrated in FIG. 5. The apparatus 500 may be comprised of a memory 505 and a database 510 both coupled to the processor 515. The memory 505 is used to store the information from the inputs 520 which is the plurality of inputs 400. The database 510 contains average male and female population information 410. The processor 515 selects the male or female specific equations 420 and executes the calculation for the BMI_{c} 430. The output 530 provides the subject with the BMI_{c}.

What is claimed is:

1. A method of calculating a corrected body mass index of a subject with a computing system comprising the steps of
obtaining said subject's gender as male or female, mass, height, waist size, body temperature, average outside temperature, room temperature, and hours spent outside per day for storage in memory of computing system;
selecting male or female formulations from said subject's gender from said computing system database;
calculating a body mass index from said subject's mass and height by said computing system processor;
calculating an average population waist to height ratio from said body mass index by said computing system processor;
adjusting an average population waist size with said average population waist to height ratio and said waist size by said computing system processor;
computing an average percent body fat from said subject's selected gender from said subject mass and average population waist size by said computing system processor;
calculating a percent body fat from said subject's said waist size and mass by said computing system processor;
processing said time spent outside to evaluate a time fraction by said computing system processor;
calculating from said time fraction, body temperature, outside temperature, and room temperature a hot or cold climate's correction factor by said computing system processor;
computing said corrected body mass index wherein said body mass index, gender's average percent body fat, subject's percent body fat, and hot or cold climate's correction factor by said computing system processor for output.

2. A method of calculating a male's corrected body mass index with a computing system from a subject comprising the steps of
selecting a male's average percent body fat formulation
$${P}_{o} ( M ) = 100 \frac{( - 98.42 + 4.15 h ( \frac{m}{h} ( 0.014 ) + 0.15 ) - 0.082 m )}{m}$$
with said male subject's waist size (W), mass (m), and height (h) by said computing system;
selecting said male subject's percent body fat
$${P}_{1} ( M ) = 100 \frac{( - 98.42 + 4.15 W - 0.082 m )}{m}$$
with said male subject's waist size (W) and said mass by computing system;
obtaining said male subject's body temperature (T_{B}), a room temperature (T_{ES}) an outside average temperature (T), a time fraction spent outsize by said male subject (β);
computing a climate's correction factor
$${T}^{′} = \frac{1}{( 1 + β ( ( \frac{{T}_{B} - T}{{T}_{B} - {T}_{ES}} ) - 1 ) )}$$
from obtained said male subject's body temperature, room temperature, outside temperature, and time fraction spent outside wherein said male subject's corrected body mass index (BMI_{c}(M)) is computed
$${BMI}_{C} ( M ) = \frac{m}{h} \frac{{T}^{′} ( \frac{W}{h ( \frac{m}{h} ( 0.014 ) + 0.15 )} )}{( \frac{1}{1.01 \frac{lbs}{{in}^{3}}} ) ( 1 - \frac{( {P}_{1} ( M ) - {P}_{0} ( M ) )}{100} ( 0.22 \frac{lbs}{{in}^{3}} ) )}$$
with said male subject's mass, height, male's average percent body fat, mate subject's percent body fat, and climate's correction factor.

3. A method of calculating a female's corrected body mass index with a computing system from a subject comprising the steps of
selecting a female's average percent body fat formulation
$${P}_{o} ( F ) = 100 \frac{( - 76.76 + 4.15 h ( \frac{m}{h} ( 0.014 ) + 0.15 ) - 0.082 m )}{m}$$
with said female subject's waist size (W), mass (m), and height (h) by said computing system;
$${P}_{1} ( F ) = 100 \frac{( - 76.76 + 4.15 W - 0.082 m )}{m}$$
selecting said female subject's percent body fat with said female subject's waist size (W) and said mass by computing system;
obtaining said female subject's body temperature (T_{B}), a room temperature (T_{ES}), outside average temperature (T), a time fraction spent outsize by said male subject (β);
computing a climate's correction factor
$${T}^{′} = \frac{1}{( 1 + β ( ( \frac{{T}_{B} - T}{{T}_{B} - {T}_{ES}} ) - 1 ) )}$$
from obtained said female subject's body temperature, room temperature, outside temperature, and time fraction spent outside wherein said female subject's corrected body mass index (BMI_{c}(F)) is computed
$${BMI}_{C} ( M ) = \frac{m}{h} \frac{{T}^{′} ( \frac{W}{h ( \frac{m}{h} ( 0.014 ) + 0.15 )} )}{( \frac{1}{1.01 \frac{lbs}{{in}^{3}}} ) ( 1 - \frac{( {P}_{1} ( F ) - {P}_{0} ( F ) )}{100} ( 0.22 \frac{lbs}{{in}^{3}} ) )}$$
with said female subject's mass, height, female's average percent body fat, female subject's percent body fat, and climate's correction factor.
